(19) United States
(12) Patent Application Publication
(10) Pub. No.: US 2008/0257347 A1
(43) Pub. Date: Oct. 23, 2008

(54) Respiratory Assistance Device

(75) Inventor: Gilbert Grasmuck, Colomiers (FR)

Correspondence Address: EGBERT LAW OFFICES, 412 MAIN STREET, 7TH FLOOR, HOUSTON, TX, 77002, US

(73) Assignee: AIRTECHNOLOGIES S.A., Colomiers (FR)

(21) Appl. No.: 10/575,507

(22) PCT Filed: Oct. 15, 2003
(86) PCT No.: PCT/FR2003/003030

§ 371 Date: Jul. 7, 2008

(51) Int. Cl. A61M 16/00

(52) U.S. Cl. 128/204.18

(57) ABSTRACT

The high-speed centrifugal ventilation device for assisting a patient respiratory function includes a centrifugal fan which is arranged in a casing box and essentially has a wheel actuated by a driver. The wheel is provided with a volute connected to the casing box in such a way that an integral fixed assembly is formed. An elastic device is interposed between the fixed assembly and the driver provided with the wheel which is driven thereby. A dynamic assembly is formed for avoiding transmission of vibrations generated by the wheel and the driver towards the fixed assembly.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention is in the area of devices acting on the respiratory system of patients, actuating a pumping apparatus for a gaseous flux. More specifically its subject is a high-speed centrifugal ventilation device for assisting a patient's respiratory function.

### BACKGROUND OF THE INVENTION

As is well known, there are centrifugal ventilation devices for assisting a patient's respiratory function which comprise in general a centrifugal ventilator housed inside a casing and essentially consists of at least of a wheel rotating at high speed, actuated by a driving element and provided with a volute. The gaseous flux is drawn in through an inlet opening in the casing, to be expelled from this casing through an evacuation opening, after its passage into the ventilator composed of the motorized wheel/volute assembly and through the gaseous flux circulation channels which are arranged inside the casing, both upstream as well as downstream of the ventilator.

To familiarize oneself with the technological environment of the present invention one may refer to these patents: WO91/17361 (OY HIGH SPEED TECH Ltd), EP0487141 (PHILIPS), U.S. Pat. No. 4,478,216 (H. G. DUKOWSKI), DE20022295 (DRAGER MEDIZINTECHNIK GmbH), DE19904119 (DRAGER MEDIZINTECHNIK GmbH), FR2807117 (TECHNIFAN), FR2810246 (TAEMA S. A.), EP1170025 (LEVITRONIX LLC), U.S. Pat. No. 6,302,105 (DANTANARAYANA MUDITHA PRADEEP et al), GB 373.146 (JOHN FURSE BANCROFT VIDAL, STONE J & CO LTD), and US 2002/005197 (CEQIELSKI MICHAEL J et al).

A general problem to solve for these centrifugal ventilation devices lies in their soundproofing which is necessary due to the noise primarily generated by the vibrations produced by the motorization of the wheel which rotates at high speed, and by the impact of the fluid flow on the walls of the casing and in the volute. More specifically, the gaseous flow in the ventilator produces sound waves of aerodynamic origin; furthermore, the mechanical vibrations of moving parts and especially of the ventilator driver produce acoustic waves when they are transmitted to the casing walls. It becomes evident that the difficulties to overcome are to prevent the transmission of these different acoustic waves so as to limit the overall sound impact of the device.

The solutions that have been habitually proposed in this area consist on the one hand of using the quietest driver possible, and on the other of equipping the device with a sound absorbing element usually built into the casing, upstream and downstream of the ventilator. It is also common to associate arrangements relative to the adaptation of the wheel and the volute, as well as the layout of the circulation channels in order to limit the sources generating aerodynamic noise. Finally it is common in this field to equip the casing with vibration reducing means that are intercalated between its walls and the assembly formed by the volute-equipped wheel and the driver.

Generally speaking it becomes evident that the difficulties to overcome in this area reside essentially in a compromise to be found in between the adaptation of the different elements forming the device, their respective layout, the space they take up, and the cost of the assembly.

The general aim of the present invention is to propose a high-speed centrifugal ventilation device for assisting a patient's respiratory function that would be laid out so as to reduce in itself the sound impact of the driver as well as of the gaseous flux circulating through the device.

The inventive approach of the present invention consisted in its overall effort in dissociating from each other a first assembly consisting of dynamic elements subject to movement, and a second assembly consisting of the fixed elements of the device. The connection between the two assemblies, one dynamic and the other fixed, is achieved through at least one flexible element that is intercalated between them, to prevent the transmission of the vibrations produced by the dynamic assembly towards the fixed assembly, axially as well as radially to the drive shaft of the wheel.

Document U.S. Pat. No. 6,302,105 (DANTANARAYANA MUDITHA PRADEEP) describes, just as the examined invention, a centrifugal ventilator for respiratory assistance comprising a casing housing a volute which sits on top of a motor-driven wheel. This document in no way divulges, in contrast to the examined invention, a regrouping of the elements of the ventilator into two distinct assemblies of elements, dynamic one and fixed ones respectively, between which a flexible mass is intercalated to prevent the transmission of vibrations from the first to the second assembly of elements.

The document GB 373 146 (JOHN FURSE-BANCROFT) describes an ambient ventilator which comprises a frame bearing a wheel-driving motor and an element that can be assimilated to a volute.

A flexible mass is intercalated between the frame and the driving element, and not, as in the examined invention, between the volute as integral to the casing, and the driving element.

A simple transposition from the provisions of patent GB 373 146 to a ventilator of U.S. Pat. No. 6,302,105 would not lead to a ventilator of the examined invention, i.e., a ventilator in which the volute is integral to the casing, to form an assembly of fixed integral elements, this assembly being separated by a flexible mass of intercalation from another assembly of dynamic elements, including the driver and the wheel, so as to prevent the transmission of vibrations generated by the latter ones towards the assembly of fixed elements.

### BRIEF SUMMARY OF THE INVENTION

Not one of the known references of the state of the art, whether taken alone or in combination, describes, as primarily claimed by the examined invention, a centrifugal ventilation device housed in a casing and primarily composed of at least one wheel rotating at high speed, driven by a driver and equipped with a volute, where the volute is integral part of the casing so as to form an integral fixed assembly, while flexible means are intercalated between this fixed assembly and the driver equipped with the wheel it drives, these forming a dynamic assembly, to prevent the transmission of vibrations generated by the dynamic assembly towards the fixed assembly, in order to eventually reduce the sound impact of the device.

More accurately, and in contrast to what has become customary in this field, it is proposed to dissociate a dynamic assembly comprising the driver and the wheel on the one hand, which are the moving elements of the device, from a fixed assembly comprising the volute which has become an integral part of the casing.

The flexible element is at least intercalated between these two assemblies for connecting one to the other, the dynamic assembly being borne in suspension by the fixed assembly.

Also, the circulation channels for the gaseous flux inside the casing are lined with soundproofing material. The junction areas between the elements delimiting the internal volumes of these channels in the casing are in turn made impervious; to form an obstacle to the acoustic waves induced by the fluid circulation and to increase the soundproofing capabilities that are proper to the device proposed by the invention.

Finally, the wheel and if applicable, a flange that surrounds it are made of light materials to reduce their reaction time at any change of the rotation speed, but without requiring significant power of the driver which generates noise and consumes energy.

More specifically the device of the present invention comprises a centrifugal ventilator housed inside a casing while being organized in the general aforementioned manner of the previous state of the art.

According to the present invention such a device is recognizable insofar as it comprises the following arrangements, taken individually or in combination.

According to a first aspect of the present invention, the volute is integral to the casing so as to form an integral fixed assembly, whereas flexible means are intercalated between this fixed assembly and the driver equipped with the wheel it drives forming a dynamic assembly. These flexible means constitute the means for preventing the transmission of vibrations generated by the dynamic assembly of the device, driver and wheel in particular, towards the fixed assembly of the device, volute and casing in particular.

In the executed example of the invention only one wheel is provided in the volute. It is however possible to provide, without going outside of the invention, to associate two wheels in the same volute or each wheel in its own volute.

These provisions are such that the dynamic elements of the device, the vibration generators, are isolated from the fixed elements of the device, the static ones. This isolation is obtained through the intermediary of flexible means which constitute means of filtering the high vibratory frequencies of the dynamic assembly to the fixed assembly.

Analog hereto in their generality, the flexible means intercalated between the dynamic assembly and the fixed assembly are for instance formed by mechanical springs of wire, blade or elastic washer type or analog, or for yet another example and preferably by a flexible material such as elastomer or foam; or also for yet another example by magnetic repulsion means.

According to a preferred realization of the invention the intercalated flexible means between the dynamic assembly and the fixed assembly comprise at least on first flexible element intercalated between the volute and the dynamic assembly and at least a second flexible element intercalated between the driver, at least at its base, and the casing.

These flexible intercalated elements also and advantageously constitute not only a mean of connection between the fixed assembly and the dynamic assembly, but also a mean for positioning the latter inside the casing.

Preferentially the flexible element intercalated between the volute and the driver is in axial and radial intercalation, whereas the flexible element intercalated between the driver and the casing is only in radial intercalation.

These flexible intercalated elements are more particularly made of elastomers the hardness, volume and conformation characteristics of which confer upon them among other things resonance frequencies in the order of 10 Hz to 300 Hz.

As an indication and for example, for excitation frequencies of the dynamic assembly comprised between 160 Hz and 20,000 Hz, the resonance frequency of the suspended assembly consisting of the dynamic assembly and flexible intercalated elements is in the order of 80 Hz.

According to a second aspect of the present invention the circulation channels for gaseous flux inside the casing are lined with a mass of soundproofing material such as open cell polyurethane foam in order to attenuate the acoustic transmissions in the gaseous flux circulation channels.

Incidentally and according to advantageous forms of realization, taken separately or in combination, any one at least of the gaseous flux circulation channels arranged inside the casing upstream and downstream of the volute:
a) is arranged around the driver in view of its cooling by the passage of the gaseous flux in its proximity;
b) is organized as a baffle; and
c) is arranged inside the casing by partitioning the latter. These partitions are for example recessed and fixed in its interior space through the intermediary of an impervious material such as silicone, forming a tight seal against the passage of acoustic waves.

According to a third aspect of the present invention the material the wheel is made of is a light material such as a plastic material or a composite material of plastic matter and mineral fibers or perhaps a cellular material, in particular a rigid foam, so as to provide it with low inertia in rotation.

Preferably the wheel is equipped with a flange of low density rigid cellular foam attached to it by glue.

One will notice that analog hereto the wheel is indifferently of the centrifugal wheel type with radial blades or a centrifugal wheel with axial input and radial output.

Likewise, one will notice that analog hereto, the driver is indifferently a direct current brush collector motor or an asynchronous motor or also a synchronous motor.

The driver is, if applicable, equipped with sensors detecting the angular position of the rotor. However, and according to another aspect of the invention, the driver is a synchronous motor with permanent magnets at the rotor, without position sensors, its operation being dependent on electronic means with vectorial control of the flow.

According to preferred arrangements of the device of the invention it includes two intakes of gaseous flux circulation that are arranged in proximity of the evacuation orifice the casing is equipped with.

One of these intakes is intended to measure the fluid pressure at the output of the casing, the other intake in turn being intended to permit the injection of oxygen to enrich the gaseous mixture which is delivered to the patient.

### BRIEF DESCRIPTION OF THE SEVERAL VIEWS OF THE DRAWINGS

The present invention will be better understood and details highlighting it will appear at the description which is going to be made of a preferred form of realization, in connection with the figures of the attached page.

FIG. 1 is a schematic longitudinal section view illustrating a device of the invention according to a preferred example of realization of the invention.

FIG. 2 is a schematic cross section view of a device illustrated in the preceding figure.

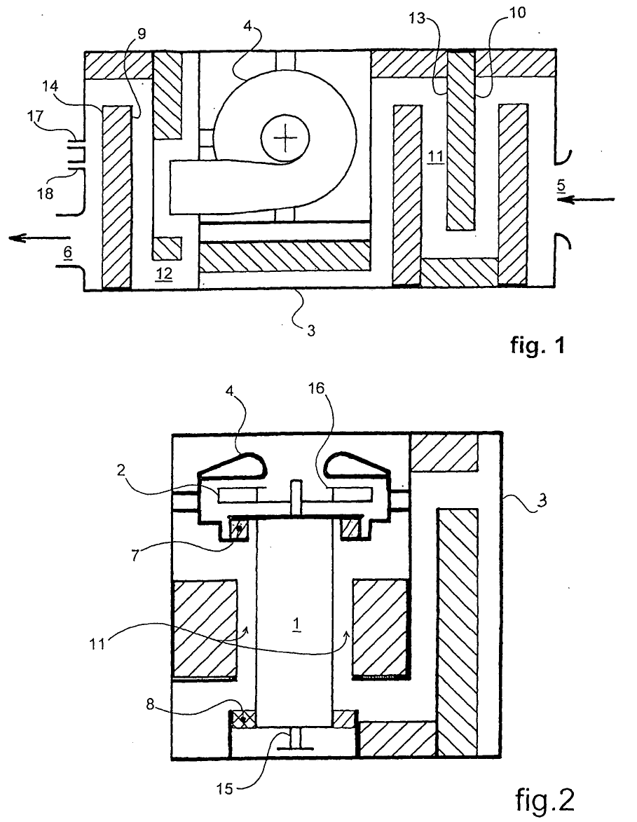

### DETAILED DESCRIPTION OF THE INVENTION

In the figures a high-speed centrifugal ventilation device is especially organized for an application to assist a patient in his respiratory function.

This device comprises in its overall form a dynamic assembly comprising a driver 1 driving a wheel 2, and a fixed assembly composed of integrated static elements, comprising a casing 3 and a volute 4 arranged around the wheel, the volute 4 being attached to the casing 3 by fitting, accessorily completed by bonding for example.

The volute 4 is intended to guide the gaseous flow drawn in by the wheel 2 from the exterior through an intake opening 5 of this gaseous flow through the casing 3, to be expelled through an output opening 6 of this gaseous flow outside of the casing 3.

Flexible elements in elastomer 7, 8 are intercalated between the fixed assembly 3, 4 and the dynamic elements 1, 2 of the device. More precisely these elements comprise a first flexible element intercalated between the volute 4 and the driver 1 at its top, and a second flexible element intercalated between the casing 3 and the driver 1, at its base.

The casing 3 is internally partitioned, by partitions such as 9 and 10, to arrange channels 11 and 12 for circulating the gaseous flow, respectively upstream and downstream of the volute 4. These channels 11, 12 are more precisely organized as baffles and are covered with a foam coating, such as 13 and 14 which line the partitions 9, 10. Also, these partitions 9, 10 are recessed and attached inside the casing 3 by glueing and in particular by sealed joints that are impervious to the passage of acoustic waves.

One will notice that a part at least of the upstream channels 11 are arranged around the driver 1, in order to participate in its cooling and in the desired sound absorption.

One will observe that the flange 16 provided on wheel 2 to surround the blades it comprises is attached to it by glueing and is made of a light material such as rigid foam.

One will also observe the presence oftwo air inlets 17 and 18 arranged across the outside wall 18 of the casing 3 in proximity to the evacuation opening 6. These inlets 17 and 18 are respectively intended to allow a measurement of the fluid pressure inside casing 3 and allow the injection of oxygen to enrich the gaseous mixture delivered to the patient.

Finally, one will notice that the motor 1 comprises, at the shaft end opposite the wheel 2, a flyweight 15 allowing dynamic balancing of the rotor of the ventilator equipped with wheel 2.

I claim:

1. High-speed centrifugal ventilation device for assisting a patient's respiratory function, the device comprising:
a centrifugal ventilator housed inside a casing and comprised of:
at least one wheel rotating at a speed driven by a driver and being equipped with a volute, and
channels circulating a gaseous flux, said channels being arranged inside said casing upstream and downstream of the ventilator, induction and discharge of said gaseous flux through openings of said casing for intake and expulsion respectively, said volute being integral part of said casing so as to form an integral fixed assembly; and
a flexible means intercalated between the fixed assembly and said driver, forming a dynamic assembly to prevent transmission of vibrations generated by said dynamic assembly towards the fixed assembly.

2. Device as per claim 1, wherein the flexible intercalated means between the dynamic assembly and the fixed assembly is comprised of at least one mechanical spring, flexible material, or magnetic repulsion means.

3. Device as per claim 2, wherein said flexible means is intercalated between the dynamic assembly and the fixed assembly for connection to each other, with the dynamic assembly being supported in suspension by the fixed assembly.

4. Device as per claim 3, wherein said flexible means intercalated between the dynamic assembly and the fixed assembly comprise:
at least one first flexible element intercalated between said volute and the dynamic assembly, and
at least one second flexible element intercalated between the driver, at least at a base thereof, and said casing.

5. Device as per claim 4, wherein the first and second flexible elements are comprised of:
connecting means between the fixed assembly and the dynamic assembly; and
positioning means for the dynamic assembly inside said casing.

6. Device as per claim 4, wherein the first and second flexible elements are comprised of elastomer material, said material having a hardness, volume and conformation providing the first and second flexible elements with resonance frequencies in a range of 10 Hz to 300 Hz.

7. Device as per claim 1, wherein said channels for gaseous flow inside said casing are lined with a mass of sound-absorbing material.

8. Device as per claim 1, wherein at least one channel, of said channels circulating gaseous flow arranged inside the casing upstream and downstream of the volute, is arranged around the driver, cooling said driver through a passage of gaseous flow in proximity of said driver.

9. Device as per claim 1, wherein at least one channel, of said channels circulating gaseous flow arranged inside the casing upstream and downstream of the volute, is organized in a form of a baffle.

10. Device as per claim 1, wherein at least one channel, of said channels circulating gaseous flow upstream and downstream of the volute, is arranged inside said casing by partitioning said casing with partitions, said partitions being recessed and attached in an inside space thereof through an intermediary of an impervious material, forming a seal against passage of acoustic waves.

11. Device as per claim 1, wherein said at least one wheel is comprised of material for low inertia in rotation.

12. Device as per claim 1, wherein said at least one wheel is equipped with a flange comprised of rigid low density foam, attached to the wheel by glueing.

13. Device as per claim 1, wherein said driver is equipped with sensors detecting angular position of a rotor.

14. Device as per claim 1, wherein said driver is comprised of a synchronous motor with permanent magnets at a rotor, without position sensors, operation thereof being put under dependence of electronic means with vectorial control of flow.

15. Device as per claim 1, further comprising:
two intakes of gaseous flux circulation, arranged in proximity of an evacuation orifice of said casing, one of said two intakes being intended to measure fluid pressure at an output of said casing, a remaining one of said two intakes being intended to permit injection of oxygen to enrich a gaseous mixture which is delivered to a patient.

16. Device as per claim 1, wherein each wheel is equipped with a volute.

17. Device as per claim 1, wherein a single volute covers several wheels.
